(19) United States
(12) Patent Application Publication
(10) Pub. No.: US 2024/0115532 A1
(43) Pub. Date: Apr. 11, 2024

(54) USE OF L-ERGOTHIONEINE FOR ALLEVIATING AND PREVENTING AGE-RELATED VISUAL DEGENERATION

(71) Applicant: NANJING NUTRABUILDING BIO-TECH CO., LTD., Nanjing (CN)

(72) Inventors: Ronghua YI, Nanjing (CN); Kylin LIAO, Nanjing (CN)

(21) Appl. No.: 18/392,229

(22) Filed: Dec. 21, 2023

Related U.S. Application Data
(63) Continuation of application No. PCT/CN2022/100009, filed on Jun. 21, 2022.

(30) Foreign Application Priority Data
Jun. 22, 2021 (WO) PCT/CN2021/101541

(51) Int. Cl. A61K 31/198; A23L 33/175; A61P 27/02

(52) U.S. Cl. CPC A61K 31/198; A23L 33/175; A61P 27/02

(57) ABSTRACT

The present invention provides methods and compositions for alleviating and preventing age-associated visual degeneration in a mammal, comprising administrating to the mammal in need thereof an effective amount of L-Ergothioneine, an analog or derivative thereof, or a pharmaceutically acceptable salt, acid, ester, polymer, analog or derivative thereof, as an active ingredient. In particular, the administration of L-Ergothioneine is capable of controlling subretinal choroidal neovascularization (CNV) level in the mammal's retina; activating Nrf2-mediated antioxidant genes to scavenge chronic reactive oxygen species and decrease vascular endothelial growth factor (VEGF) level in the mammal's retinal pigment epithelium (RPE); activating Nrf2-mediated antioxidant genes by lowering the damage induced by oxidative stress (OS) in the mammal's RPE; and/or inhibiting oxidation-induced epithelial-to-mesenchymal transition (EMT) to prevent epithelial fibrosis in RPE in mammals' RPE.

### CROSS REFERENCE TO RELATED APPLICATION

This application is a continuation application of International Patent Application No. PCT/CN2022/100009, filed on Jun. 21, 2022, which claims the priority of the International Application No. PCT/CN2021/101541, filed on Jun. 22, 2021, the contents of all of which are incorporated herein by reference in their entirety.

### FIELD OF THE INVENTION

This invention generally relates to the field of methods for alleviating and preventing age-related (age-associated) visual degeneration in a mammal, and more specifically relates to methods comprising administrating to a mammal in need thereof: L-Ergothioneine, an analog or derivative thereof, or a pharmaceutically acceptable salt, acid, ester, polymer, analog or derivative thereof, as an active ingredient, in order to alleviate and/or prevent age-related (age-associated) visual degeneration in the mammal.

### BACKGROUND OF THE INVENTION

With the global aging population, senescence and degeneration of ocular organ function in seniors, especially over age of 40, has become a common and important issue, which would affect individual lives and increase social burden. The elderly, age-induced or age-associated senescence and degeneration of ocular organ function is also called age-related visual degeneration. The age-related visual degeneration may damage outer retina, such as retinal pigment epithelium (RPE, an important role for absorbing light), Bruch's membrane (BM), the underlying choroid (choriocapillaris) and photoreceptors. What's worse, the age-related visual degeneration may lead to blurred vision, retinal hemorrhage, macular oedema, fibrosis, drusen deposits, BM thickening, subretinal choroidal neovascularization (CNV) and even ultimately blindness. Therefore, the age-related visual degeneration not only decreases the seniors' quality of life due to the resulting restricted autonomy, mobility and activities of living, but also cause negative social and psychological effects.

There are several corresponding factors or reasons associated with the age-related visual degeneration. First, the age-related visual degeneration may induce high levels of cytokines (e.g., vascular endothelial growth factor (VEGF) and elderly's retinas, especially elderly's RPE); influence the inflammatory state; and lead to increased apoptosis and thus to the progression of visual decline. Second, nuclear factor erythroid 2-related factor 2 (Nrf2), which is a master transcription factor to activate the coordinated system of endogenous antioxidant and anti-inflammatory protection, can mediate oxidative stress (OS) response signaling pathway, and is important in the prevention of oxidative damage. The Nrf2 has been reported to be associated with the risk of the age-related visual degeneration, as it has potential capacity in the protection of RPE cells from OS-related damages and cell death, and thus it could be beneficial in ocular health. See, e.g., Sachdeva, M. M., M. Cano, and J. T. Handa, Nrf2 signaling is impaired in the aging RPE given an oxidative insult. Experimental Eye Research, 2014. 119: p. 111-114. That said, the antioxidant function of Nrf2 may decrease as the individual is aged, such that the aging RPE is vulnerable to OS and then its functional activity declines and its capacity neutralize reactive oxygen species (ROS) is reduced. A single knock-out animal model of Nrf2 genie has been shown to induce RPE degeneration—displayed as accumulation of lipofuscin and drusen-deposits, choroidal neovascularization and increase of inflammatory proteins in RPE, as well as accumulation of autophagy-related bodies. See, e.g., Zhao, Z., et al., Age-Related Retinopathy in NRF2-Deficient Mice. PLOS ONE, 2011. 6(4): p. e19456. Third, the epithelial-to-mesenchymal transition (EMT) represents a change in cellular phenotype—i.e., the cell changing from being polarized, layered and immobile to a non-layered, spindle-like cell, which is capable of migration due to high resistance to stress and apoptotic cell death, thereby contributing to the fibrosis encountered in RPE.

By far, while there have been some drugs developed to relieve these symptoms, the existing drugs have several side effects, such as damaging human liver and kidney function. The conventional nutritional methods of attenuating the age-related visual degeneration mainly protect eyesight seemingly to the small extend, and the effects of those approaches are not satisfactory at present.

L-Ergothioneine is a water-soluble amino and antioxidant, found mainly in mushrooms, but also in king crab, meat from animals that have grazed on grasses containing L-Ergothioneine, and other foods. L-Ergothioneine cannot be synthesized by the human body (or other vertebrates), as it can only be supplemented by diet. It is a naturally occurring thiol/thione derivative of the essential amino acid histidine, and its molecular formula is C₉H₁₅N₃O₂S—combined with hydrogen, that sulfur at the end is what actually classifies it as a “thiol”, the “—SH group”, is either referred to as a thiol group or a sulfanyl group.

In view of the drawbacks of conventional methods of attenuating age-related visual degeneration, there remains a significant and urgent need for a novel strategy for alleviating and preventing the ageing and senescence of retina and macula. It is particularly desired to have a substance to effectively and fundamentally ameliorate and prevent age-associated visual degeneration.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

The present invention generally relates to methods for alleviating and preventing age-associated visual degeneration in a mammal, comprising administrating to the mammal in need thereof an effective amount of L-Ergothioneine, an analog or derivative thereof, or a pharmaceutically acceptable salt, acid, ester, polymer, analog or derivative thereof, as an active ingredient.

Particularly, L-Ergothioneine is surprisingly found to be a powerful substance capable of effectively and fundamentally ameliorating and preventing age-associated visual degeneration. L-Ergothioneine is concentrated in cells and tissues wherein exposed to OS frequently, such as ocular tissues (lens, retina, cornea and RPE), liver, bone marrow and seminal fluid. L-Ergothioneine (C₉H₁₅N₃O₂S) is tautomeric and exists predominantly in the thione form in neutral aqueous solutions. Moreover, L-Ergothioneine with sulfhydryl group (—SH) has an extraordinarily antioxidant role via many pathways, such as the constitutive activation of Nrf2-mediated antioxidant genes by lowering the damage induced by OS and the inhibition of oxidation-induced EMT. As such, L-Ergothioneine has potential benefits in the protection of RPE cells from fibrosis and OS-related damages and cell death, therewith the VEGF level being reduced in retina. According to this invention, it was surprisingly found that L-Ergothioneine can alleviate and prevent age-related visual degeneration effectively at root in the aging human by controlling CNV, thereby activating Nrf2-mediated antioxidant genes, downregulating VEGF level and inhibiting oxidation-induced EMT. It is believed that this invention is the first time to propose and conduct L-Ergothioneine as an active ingredient to alleviate and prevent age-related visual degeneration.

One aspect of this invention relates to a method for alleviating and preventing age-associated visual degeneration in a mammal, comprising administrating to the mammal in need thereof an effective amount of L-Ergothioneine, an analog or derivative thereof, or a pharmaceutically acceptable salt, acid, ester, polymer, analog or derivative thereof, as an active ingredient.

In some embodiments, the age-related visual degeneration is related to phenomenon comprising eyestrain, blurred vision, dry eyes, eye fatigue, or blindness.

In some embodiments, the age-related visual degeneration is characterized by retinal effusion, retinal hemorrhage, macular oedema, accumulation of lipofuscin and drusen-deposits, Bruch's membrane (BM) thickening, subretinal choroidal neovascularization (CNV) and/or increase of inflammatory proteins in retinal pigment epithelium (RPE).

In some embodiments, the administration of L-Ergothioneine is capable of controlling choroidal neovascularization (CNV) level in the mammal's retina.

In some embodiments, the administration of L-Ergothioneine is capable of activating Nrf2-mediated antioxidant genes to scavenge chronic reactive oxygen species and decrease vascular endothelial growth factor (VEGF) level in the mammal's RPE.

In some embodiments, the administration of L-Ergothioneine is capable of activating Nrf2-mediated antioxidant genes by lowering the damage induced by oxidative stress (OS) in the mammal's RPE.

In some embodiments, the administration of L-Ergothioneine is capable of inhibiting oxidation-induced epithelial-to-mesenchymal transition (EMT) to prevent epithelial fibrosis in RPE in the mammals' RPE.

In some embodiments, the mammal is an old mammal. In some embodiments, the mammal is a human or an animal (e.g., pet or cattle). For instance, the mammal is a human is aged 30 years or older (e.g., 40 years, 60 years, or 70 years).

In some embodiments, L-Ergothioneine is administrated in a nutritional, drinking, pabulary or pharmaceutical composition in a food, drink, nutritional, or pharmaceutical field.

In some embodiments, L-Ergothioneine is administrated orally, by intravenous injection, by intramuscular injection, intraperitoneally or sublingually.

In some embodiments, L-Ergothioneine is administrated in a form of solutions, liquid suspensions, parenteral solutions, injections, tablets, pills, granules, powder, film, (micro)capsules, aerosols, tonics, syrups, beverages, nourishments, snack bar or a food composition.

In some embodiments, L-Ergothioneine is administrated by oral with a daily dose ranging from 2 to 2000 mg. The daily dose may be administrated by a single dose or multiple divided doses.

In some embodiments, L-Ergothioneine is administrated at least once or multiple times a day. In some embodiments, L-Ergothioneine is administrated daily for at least seven days in one period.

Another aspect of the present invention provides a use of L-Ergothioneine for preparing a composition for effectively alleviating and preventing age-associated visual degeneration in a mammal, wherein the composition comprises an effective amount of L-Ergothioneine, an analog or derivative thereof, or a pharmaceutically acceptable salt, acid, ester, polymer, analog or derivative thereof, as an active ingredient.

In some embodiments, L-Ergothioneine is prepared in a form of solutions, liquid suspensions, parenteral solutions, injections, tablets, pills, granules, powder, film, (micro)capsules, aerosols, tonics, syrups, beverages, nourishments, snack bar or a food composition.

In some embodiments, the composition comprises L-Ergothioneine with a dose ranging from 2 to 2000 mg

In some embodiments, L-Ergothioneine in the composition is capable of (i) controlling CNV level in the mammal's retina; (ii) activating Nrf2-mediated antioxidant genes to scavenge chronic reactive oxygen species and decrease VEGF level in the mammal's RPE; (iii) activating Nrf2-mediated antioxidant genes by lowering the damage induced by OS in the mammal's RPE; and/or (iv) inhibiting oxidation-induced EMT to prevent epithelial fibrosis in RPE in mammals' RPE.

In some embodiments, the age-related visual degeneration is related to phenomenon comprising eyestrain, blurred vision, dry eyes, eye fatigue, or blindness.

Still in some embodiments, the age-related visual degeneration is characterized by retinal effusion, retinal hemorrhage, macular oedema, accumulation of lipofuscin and drusen-deposits, BM thickening, subretinal choroidal neovascularization (CNV) and/or increase of inflammatory proteins in RPE.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “effective amount” refers to an amount that is required to improve at least one symptom of a medical condition in an individual.

### BRIEF DESCRIPTION OF THE DRAWINGS

FIG. 1 illustrates the choroidal vessel area of the zebrafish eye after sample processing (Compared with model control group, *p<0.05, *** P<0.001).

FIG. 2 is a typical picture of the histopathological section of the zebrafish eye after sample processing.

FIG. 3 is a ROS fluorescence value of zebrafish after sample processing (Compared with model control group, ***p<0.001).

FIG. 4 is the effect of L-Ergothioneine administration on the relative expression level of VEGFR1 genes (compared with model control group, ** P<0.01, *** P<0.001).

FIG. 5 is the effect of L-Ergothioneine administration on the relative expression level of hif-1α genes (compared with model control group, ** P<0.01, *** P<0.001).

FIG. 6 is the effect of L-Ergothioneine administration on the relative expression level of vegfaa genes (compared with model control group, *p<0.05, **p<0.01).

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for methods for administrating L-Ergothioneine (or an analog or derivative thereof, or a pharmaceutically acceptable salt, acid, ester, polymer, analog or derivative thereof) as the active ingredient to a mammal (e.g., a senior) for effectively alleviating and preventing age-associated visual degeneration in a mammal. For instance, the age-related visual degeneration may include phenomenon such as eyestrain, blurred vision, dry eyes, eye fatigue, and/or blindness phenomenon. The age-related visual degeneration may also be characterized by retinal effusion, retinal hemorrhage, macular oedema, accumulation of lipofuscin and drusen-deposits, BM thickening, subretinal choroidal neovascularization (CNV) and/or increase of inflammatory proteins in RPE. Particularly, L-Ergothioneine is capable of (i) controlling CNV level in the mammal's retina; (ii) activating Nrf2-mediated antioxidant genes to scavenge chronic reactive oxygen species and decrease VEGF level in the mammal's RPE; (iii) activating Nrf2-mediated antioxidant genes by lowering the damage induced by OS in the mammal's RPE; and/or (iv) inhibiting oxidation-induced EMT to prevent epithelial fibrosis in RPE in mammals' RPE. Moreover, L-Ergothioneine may be administrated in a variety of forms, such as solutions, liquid suspensions, parenteral solutions, injections, tablets, pills, granules, powder, film, (micro)capsules, aerosols, tonics, syrups, beverages, and nourishments. For instance, L-Ergothioneine may be administrated (e.g., by oral) with a daily dose ranging from 2 to 2000 mg for at least seven days in one period. Further, the present invention also provides use of L-Ergothioneine for preparing a composition for effectively ameliorating and preventing age-associated visual degeneration in a mammal, wherein the composition comprises an effective amount of L-Ergothioneine, an analog or derivative thereof, or a pharmaceutically acceptable salt, acid, ester, polymer, analog or derivative thereof, as an active ingredient.

The following examples are illustrative of select embodiments of the present invention and are not meant to limit the scope of the invention.

### EXAMPLES

The healthy and male C57BL/6J mice at 18-24 months of age were maintained under the barrier conditions in a temperature-controlled environment and fed a commercial mouse chow.

The healthy and male C57BL/6J mice at 14-18 months of age were maintained under the barrier conditions in a temperature-controlled environment and fed a commercial mouse chow.

Animal mode of subretinal choroidal neovascularization (CNV): All mice were subretinally injected with Matrigel in both eyes under general anesthesia. The liquid Matrigel (75% v/v) was prepared using phosphate-buffered saline (PBS) at 4° C. After exposing the sclera, an incision was made between the limbus and the equator using a sharp No. 33 size needle. The Matrigel suspension (0.5 μl per eye) administered via a Hamilton microsyringe with a No. 33 blunt tip needle was slowly injected into the subretinal space along the previous needle tract. Following injection, the Matrigel polymerized to form a solid gel. Fluorescence angiograms were performed over a period of 3 weeks to identify whether mice have CNV.

Fluorescein angiography: After subretinal injection, fluorescein angiograms were obtained on days 3, 7, 10, 14, and 21. Briefly, every mouse group (n=5) were anesthetized with ketamine and xylazine, and the pupils were dilated with 0.5% tropicamide eye drops. Next, 0.1 ml sodium fluorescein (25%) was injected intraperitoneally, and serial angiograms were captured at approximately 3 to 10 min after dye injection using a Nikon D1 (Nikon) digital camera (16×magnifiers, power supply flash 3) attached to a Zeiss photo-slit-lamp. Vascular leakage (early symptom of CNV) was defined as the presence of a hyperfluorescent spot that increased in size over time.

They were randomly divided into three groups: (1) non-supplement group (Con, n=6); (2) L-Ergothioneine supplement group with low dosage (L-Ergothioneine, 2 mg/kg/d, n=6); (3) L-Ergothioneine supplement group with high dosage (L-Ergothioneine, 10 mg/kg/d, n=6). Some mice were administered L-Ergothioneine for once daily by gavage in normal saline solution for 5 months.

### Test 1. Controlling CNV Level

Before all mice were euthanized, mice were measured by Fluorescein angiography to prove the alleviating level of CNV phenomena by L-Ergothioneine, compared with test results before.

The result shows that CNV level was attenuated obviously by L-Ergothioneine.

### Test 2. Activating Nrf2-Mediated Antioxidant Genes to Scavenge Chronic Reactive Oxygen Species and Downregulating VEGF Level in Retina

Mice were euthanized by CO2 inhalation followed by cervical dislocation at the end of study and their eyes were immediately removed. The eyes of some mice (n=3) fixed in 2% paraformaldehyde for 2 h and the retina samples of some mice (n=3) were isolated from eyes radically. All tissues were frozen in optimal cutting temperature embedding compound.

Partial retina tissues were obtained and rapidly broken up in ice-cold normal saline. A tissue homogenate (10%, w/v) was then prepared.

Antioxidant capacity of the retina was measured using an assay based on the ability of the sample to inhibit the oxidation of 2,2′-azino-di-[3-ethylbenzthiazoline sulfonate]+(ABTS) by metmyoglobin. The amount of ABTS+ produced was measured in terms of the decrease in absorbance caused by the antioxidants in the sample. Reactive oxygen species (ROS) were quantified by fluorescence spectroscopy using 2′,7′-dichlorofluorescein diacetate. Testing antioxidant capacity and ROS to prove the L-Ergothioneine antioxidant ability.

Total RNA was isolated from retina tissues using Trizol reagent. Real-time PCR was measured and performed with the following primers: Nrf2 forward 5′-CCGCCCTCCATATGATGGACTTGGA-3′. RNA samples were reverse-transcribed to cDNA and subjected to quantitative PCR, performed with a Light Cycler 96 Real-Time PCR System using the AceQ qPCR SYBR Green Master Mix. Nrf2 measurements can be identify the capacity of L-Ergothioneine-mediated Nrf2.

Retina tissues homogenate was centrifuged (2000 rpm, 10 min, 4° C.) and the supernatants were collected and applied to a pre-coated human VEGF microplate. VEGF concentrations was measured in experimental groups of mice using enzyme-linked immunosorbent assay (ELISA) kits to evaluate the levels of proinflammatory cytokines in the retina. This test can assess that the ability of L-Ergothioneine to prevent age-related visual degeneration-induced increases in VEGF level.

The result shows that L-Ergothioneine activated Nrf2-mediated genes in retina, to scavenge chronic ROS by about 45% and decrease the VEGF level significantly by about 50%.

### Test 3. Inhibiting Oxidation-Induced EMT to Prevent Epithelial Fibrosis in RPE

From paraffin embedded blocks, 3 μm-thick parasagittal serial sections were cut with a microtome for future testing the immunoreactivity of mesenchymal markers. The immunoreactivity of mesenchymal markers (Slug, Snail, vimentin, OB-cadherin) was examined with a confocal microscope. And semi-quantitative comparative densitometric analysis performed by ImageJ of the immunoreactivity of the mesenchymal markers in 1-μm parasagittal sections of RPE. Alexa Fluor secondary antibodies were used to detect specific mesenchymal markers. DAPI (4,6-diamidino-2-fenilindole, dihydrochloride) was used to stain the nuclei of RPE cells.

The result shows that L-Ergothioneine decreased inflammatory proteins and inhibited oxidation-induced EMT to prevent epithelial fibrosis obviously in RPE.

### Test 4. Mitigating Subretinal Choroidal Neovascularization

Experimental animals: Zebrafish were raised in fish water at 28° C. (water quality: 200 mg of instant sea salt was added to each 1 L of reverse osmosis water, the conductivity was 450-550 μS/cm; pH was 6.5-8.5; hardness was 50-100 mg/cm L CaCO₃). Transgenic vascular green fluorescent zebrafish (Fli-1 strain) were reproduced by natural paired mating. Age is 1 day after fertilization (1 dpf).

Sample preparation: L-Ergothioneine, white powder, was prepared into 2.00 mg/mL mother solution with standard dilution water. Positive control: Aescin and digitalis double glucoside eye drops was directly sucked the original solution.

Experimental design: The transgenic vascular green fluorescent strain zebrafish aged 1 day after fertilization (1 dpf) were randomly selected and placed in a 6-well plate, with 30 fish in each well (each experimental group). L-Ergothioneine (concentrations of 500, 1000, and 2000 μg/mL) were administered in water solution, respectively, and the positive control escin and digitalis glucoside eye drops (containing 0.3 μg/mL of digitonin and 2.0 μg/mL of escin) were administered, respectively. At the same time, the normal control group and the model control group were set, and the volume of each well was 3 mL. Except for the normal control group, the other experimental groups were given water-soluble cobalt chloride to establish the zebrafish wet macular degeneration model. After 3 days of treatment at 28° C., 10 zebrafish were randomly selected from each experimental group and placed under a fluorescence microscope to take pictures, and the NIS-Elements D 3.20 advanced image processing software was used to analyze and collect the data. Statistical analysis of the results to evaluate the efficacy of the samples to prevent retinal degeneration.

FIG. 1 illustrates the choroidal vessel area of the zebrafish eye after sample processing (Compared with model control group, *p<0.05, *** P<0.001). As illustrated, L-Ergothioneine could mitigate subretinal choroidal neovascularization.

### Test 5. Improving the Retinal Structure Damaged by Subretinal Choroidal Neovascularization

Experimental animals, sample preparation, sample processing and administration are same as test 4.

After that, these zebrafish after 3 days of administration were fixed with 4% histiocyte fixative, then stained with H&E and they were subject to test ocular histopathological analysis.

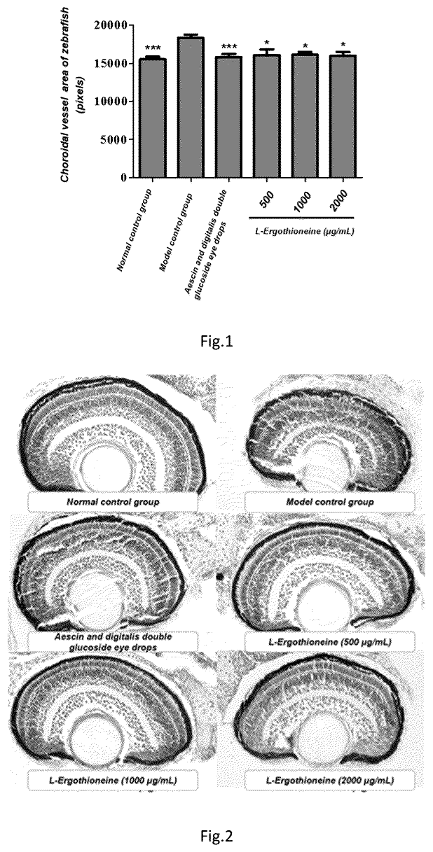

FIG. 2 are typical pictures of the histopathological section of the zebrafish eye after sample processing. As illustrated, L-Ergothioneine can improve the damage of zebrafish retinal structure caused by subretinal choroidal neovascularization. Moreover, L-Ergothioneine's effect is better than that of aescin and digitalis double glucoside eye drops.

### Test 6. Scavenging Chronic ROS to Prevent Retinal Degeneration

Except that the positive control is NAC (N-acetyl-L-cysteine, a good ROS scavenger, 4.08 μg/mL), the zebrafish are wild-type AB strain zebrafish, the concentration and sample preparation of L-Ergothioneine, sample processing are the same as in test 4.

After 2 days of supplementation at 28° C., zebrafish were stained with CM-H2DCFDA, and the original concentration in each well remained unchanged. 10 zebrafish were randomly selected from each experimental group and transferred to black 96 microtiter plates. After staining at 28° C. in the dark for 20 hours, the ROS fluorescence value was measured by a multi-function microplate reader, and the antioxidant effect of L-Ergothioneine was evaluated by the statistical analysis results of this result.

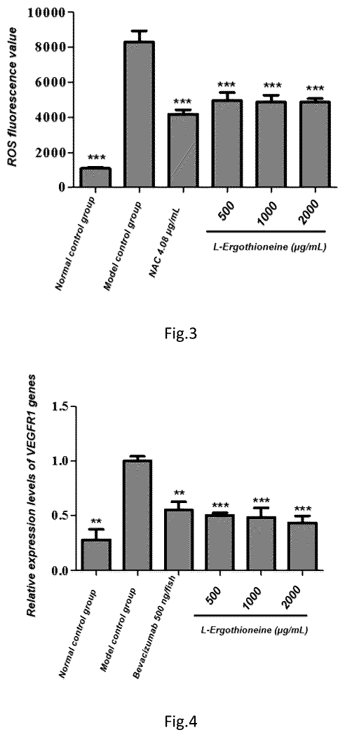

FIG. 3 is the ROS fluorescence value of zebrafish after sample processing (Compared with model control group, ***p<0.001). FIG. 3 shows that L-Ergothioneine has a good effect on preventing retinal degeneration.

### Test 7. Down-Regulating the VEGF Level by Decreasing the Expression of VEGFR1 Genes

Except that the positive control is bevacizumab (a potent antiangiogenic substance), experimental animals, sample preparation of L-Ergothioneine and sample processing are the same as in test 4.

After 3 days of supplementation at 28° C., the total RNA of each group of zebrafish was extracted using an RNA rapid extraction kit, and the concentration and purity of total RNA were determined by UV-Vis spectrophotometer. Take 2.00 μg of total RNA from zebrafish samples and follow the instructions of the first-strand cDNA synthesis kit to synthesize 20.0 μL of cDNA, and detect the expression of ß-actin and VEGFR1 genes by q-PCR. Using 6-actin as an internal reference for gene expression, the relative RNA expression levels of VEGFR1 genes were calculated.

FIG. 4 is the effect of L-Ergothioneine administration on the relative expression level of VEGFR1 genes (compared with model control group, ** P<0.01, *** P<0.001). As shown, L-Ergothioneine can down-regulate the VEGF level by decreasing the expression of VEGFR1 genes.

### Test 8. Down-Regulating the Expression of Genes Related to Visual Degeneration by Lowering the Expression Level of Hif-1α and Vegfaa Genes

The other test condition and methods are the same as test 7. Except that just detect 6-actin, hif-1α and vegfaa genes by q-PCR.

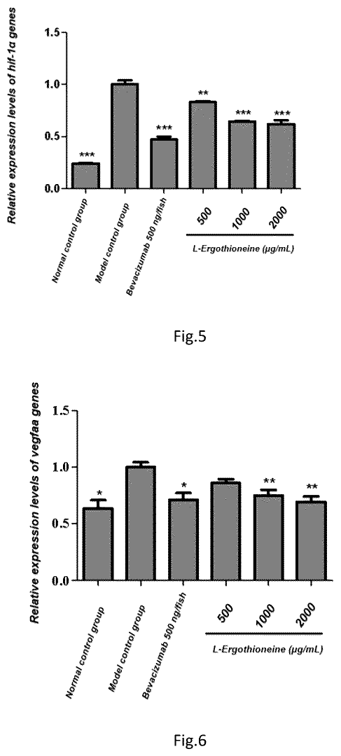

FIGS. 5 and 6 are the effects of L-Ergothioneine administration on the relative expression level of hif-1α genes (compared with model control group, ** P<0.01, *** P<0.001) and vegfaa genes (compared with model control group, *p<0.05, **p<0.01). As illustrated, L-Ergothioneine can down-regulate the expression of genes related to visual degeneration by lowering the expression level of hif-1α and vegfaa genes.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### OTHER EMBODIMENTS

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for alleviating and preventing age-associated visual degeneration in a mammal, comprising administrating to the mammal in need thereof an effective amount of L-Ergothioneine, an analog or derivative thereof, or a pharmaceutically acceptable salt, acid, ester, polymer, analog or derivative thereof, as an active ingredient.

2. The method of claim 1, wherein the age-related visual degeneration is related to phenomenon comprising eyestrain, blurred vision, dry eyes, eye fatigue, or blindness.

3. The method of claim 1, wherein the age-related visual degeneration is characterized by retinal effusion, retinal hemorrhage, macular oedema, accumulation of lipofuscin and drusen-deposits, Bruch's membrane (BM) thickening, subretinal choroidal neovascularization (CNV) and/or increase of inflammatory proteins in retinal pigment epithelium (RPE).

4. The method of claim 1, wherein the administration of L-Ergothioneine is capable of controlling choroidal neovascularization (CNV) level in the mammal's retina.

5. The method of claim 1, wherein the administration of L-Ergothioneine is capable of activating Nrf2-mediated antioxidant genes to scavenge chronic reactive oxygen species, decrease vascular endothelial growth factor (VEGF) level, hif-1α level or vegfaa level in the mammal's RPE.

6. The method of claim 1, wherein the administration of L-Ergothioneine is capable of decreasing VEGFR1 gene level, hif-1α gene level or vegfaa level in the mammal's RPE.

7. The method of claim 1, wherein the administration of L-Ergothioneine is capable of activating Nrf2-mediated antioxidant genes by lowering the damage induced by oxidative stress (OS) in the mammal's RPE.

8. The method of claim 1, wherein the administration of L-Ergothioneine is capable of inhibiting oxidation-induced epithelial-to-mesenchymal transition (EMT) to prevent epithelial fibrosis in RPE in the mammals' RPE.

9. The method of claim 1, wherein L-Ergothioneine is administrated in a nutritional, drinking, pabulary or pharmaceutical composition in a food, drink, nutritional, or pharmaceutical field.

10. The method of claim 1, wherein L-Ergothioneine is administrated orally, by intravenous injection, by intramuscular injection, intraperitoneally or sublingually.

11. The method of claim 1, wherein L-Ergothioneine is administrated in a form of solutions, liquid suspensions, parenteral solutions, injections, tablets, pills, granules, powder, film, (micro)capsules, aerosols, tonics, syrups, beverages, nourishments, snack bar or a food composition.

12. The method of claim 1, wherein L-Ergothioneine is administrated by oral with a daily dose ranging from 2 to 2000 mg.

13. The method of claim 1, wherein the daily dose is administrated by a single dose or multiple divided doses.

14. The method of claim 1, wherein L-Ergothioneine is administrated at least once or multiple times a day.

15. Use of L-Ergothioneine for preparing a composition for effectively alleviating and preventing age-associated visual degeneration in a mammal, wherein the composition comprises an effective amount of L-Ergothioneine, an analog or derivative thereof, or a pharmaceutically acceptable salt, acid, ester, polymer, analog or derivative thereof, as an active ingredient.

16. The use of claim 15, wherein L-Ergothioneine is prepared in a form of solutions, liquid suspensions, parenteral solutions, injections, tablets, pills, granules, powder, film, (micro)capsules, aerosols, tonics, syrups, beverages, nourishments, snack bar or a food composition.

17. The use of claim 15, wherein the composition comprises L-Ergothioneine with a dose ranging from 2 to 2000 mg

18. The use of claim 15, wherein L-Ergothioneine in the composition is capable of (i) controlling CNV level in the mammal's retina; (ii) activating Nrf2-mediated antioxidant genes to scavenge chronic reactive oxygen species and decrease VEGF level in the mammal's RPE; (iii) activating Nrf2-mediated antioxidant genes by lowering the damage induced by OS in the mammal's RPE; and/or (iv) inhibiting oxidation-induced EMT to prevent epithelial fibrosis in RPE in mammals' RPE.

19. The use of claim 15, wherein the age-related visual degeneration is related to phenomenon comprising eyestrain, blurred vision, dry eyes, eye fatigue, or blindness.

20. The use of claim 15, wherein the age-related visual degeneration is characterized by retinal effusion, retinal hemorrhage, macular oedema, accumulation of lipofuscin and drusen-deposits, BM thickening, subretinal choroidal neovascularization (CNV) and/or increase of inflammatory proteins in RPE.
